(19) United States
(12) Patent Application Publication
(10) Pub. No.: US 2025/0369850 A1
(43) Pub. Date: Dec. 4, 2025

(54) METHOD FOR PREDICTING DYNAMIC ADSORPTION CAPACITY OF VOLATILE ORGANIC COMPOUNDS (VOCs) AT DIFFERENT CONCENTRATIONS USING STATIC ADSORPTION ISOTHERM

(71) Applicant: University of Chinese Academy of Sciences, Huairou District (CN)

(72) Inventors: Zhongshen ZHANG, Huairou District (CN); Zhengping HAO, Huairou District (CN); Xinxin WANG, Huairou District (CN); Wenqing WU, Huairou District (CN); Zeyu ZHAO, Huairou District (CN); Xinyuan REN, Huairou District (CN); Ganggang LI, Huairou District (CN); Guoxia JIANG, Huairou District (CN); Wenpeng LI, Huairou District (CN)

(21) Appl. No.: 19/072,018

(22) Filed: Mar. 6, 2025

(30) Foreign Application Priority Data
May 31, 2024 (CN) 202410699778.X

(51) Int. Cl. G01N 7/04; G01N 33/00

(52) U.S. Cl. CPC G01N 7/04; G01N 33/0047

(57) ABSTRACT

Provided is a method for predicting a dynamic adsorption capacity of volatile organic compounds (VOCs) at different concentrations using a static adsorption isotherm. A static adsorption capacity Qₛ of the VOCs at different pressures is initially obtained, and then a dynamic penetrated adsorption capacity Q_{dp} and a dynamic saturated adsorption capacity Q_{ds} of the VOCs at the multiple concentrations are obtained. A conversion relationship equation Formula 1 between the dynamic saturated adsorption capacity Q_{ds} and the static adsorption capacity Qₛ at a same partial pressure is determined by statistics of the dynamic saturated adsorption capacity Q_{ds} and the static adsorption capacities Qₛ at the same partial pressure. A curve of the dynamic saturated adsorption capacity Q_{ds} versus the partial pressure is finally obtained according to a change trend of the static adsorption isotherm with a pressure.

### CROSS REFERENCE TO RELATED APPLICATION

This patent application claims the benefit and priority of Chinese Patent Application No. 202410699778X filed with the China National Intellectual Property Administration on May 31, 2024, the disclosure of which is incorporated by reference herein in its entirety as part of the present application.

### TECHNICAL FIELD

The present disclosure relates to the technical field of waste gas treatment, and in particular to a method for predicting a dynamic adsorption capacity of volatile organic compounds (VOCs) at different concentrations using a static adsorption isotherm.

### BACKGROUND

Volatile organic compounds (VOCs) are widely used as raw materials, solvents, and extractants in various industries such as medicine, chemicals, packaging, and printing. VOCs have high atmospheric chemical reactivity and are important precursors to the formation of fine particulate matter (PM_{2.5}) and ozone (O₃). The emission of VOCs not only causes serious pollution to the environment, but also causes direct or indirect harm to human health. Naturally, VOCs control has become the focus of current air pollution management. At present, VOCs treatment techniques mainly include adsorption, catalysis, biological treatment, thermal combustion, plasma, absorption, membrane separation, and condensation. Adsorption recovery is one of the most widely used VOCs control techniques, shows high purification efficiency and wide application range, and has been widely used in VOCs treatment and recovery.

In recent years, with the urgent need for in-depth treatment and resource utilization of VOCs pollution control, the development of adsorption recovery technology has gradually become more refined, such as developing special adsorbents with specific functions for different types of VOCs and different emissions. Therefore, although adsorption technology has been widely used, there are still many issues that need further in-depth research. The adsorption performance test of adsorbent materials is an important prerequisite and basis for the development of high-performance special adsorbents.

Commonly used adsorption performance test methods mainly include dynamic adsorption test and static adsorption test. Generally speaking, the adsorbent and the adsorbed gas are in full contact for a long time at a specific pressure to reach equilibrium during the static adsorption test. The static adsorption results at different pressures are tested to obtain the adsorption isotherms of the adsorbent to an adsorbate at different temperatures. The dynamic adsorption test generally examines an adsorption penetration curve of the adsorbent at a specific VOCs concentration (the exhaust gas contains air or other gases in addition to the VOCs) to determine the adsorption capacity of the adsorbent for specific VOCs. In comparison, dynamic adsorption is closer to practical applications and then generally used as an important basis for determining adsorbent performance in practical applications.

However, there are large differences in the concentrations of different VOCs emissions, and there are also certain fluctuations in the concentration of VOCs in many exhaust gases. Studies have shown that the concentration of VOCs in exhaust gas can have a significant impact on the adsorption capacity of the adsorbent. In addition, due to large differences in the pore structures of different adsorbent materials, their adsorption capacity may vary greatly within a specific concentration range. Accordingly, test results of the dynamic adsorption capacity of VOCs at a specific concentration cannot fully reflect the adsorption performance of the adsorbent. If the dynamic adsorption capacity of the adsorbent for VOCs of multiple concentrations is tested within a wide concentration range according to a specific concentration gradient, a heavy workload is caused and may be detrimental to the promotion and application of the technology.

Static adsorption test, a method for testing the adsorption performance of adsorbent materials for VOCs, is equally important as the dynamic adsorption test. However, studies have shown that directly comparing a saturated adsorption capacity of the static adsorption with a penetrated adsorption capacity of the dynamic adsorption cannot achieve an effective correlation relationship. The static adsorption capacity is generally assumed to be an adsorption capacity at a concentration close to the saturated vapor pressure of VOCs. The concentration of VOCs in dynamic adsorption is always much lower than the saturated vapor pressure of VOCs. The adsorption values of the two adsorption test methods are close in some adsorbent materials, but there are large differences in other adsorbent materials. This is mainly because that the pore structures of different adsorbent materials vary, resulting in different utilization rates of the pores of adsorbent materials during the static adsorption and dynamic adsorption. There is no effective correlation between the static adsorption capacity and the dynamic adsorption capacity, making it difficult for the adsorption capacities obtained by the two test methods to provide an effective reference for each other.

If there is a method that can reflect the dynamic adsorption capacity of an adsorbent material for VOCs at different concentrations through a simple static adsorption test, the workload can be effectively simplified for dynamic adsorption test of VOCs (at different concentrations). Meanwhile, if this method can make a relatively complete determination on the adsorption performance of the adsorbent material (at different concentrations), there may be an important reference for the development of efficient adsorbent materials and techniques. So far, there is no method for predicting the adsorption capacity that can be extended to dynamic adsorption at different concentrations, which to a certain extent limits the development of adsorbent materials that are specialized for the adsorption of specific VOCs and the optimization of adsorption techniques.

### SUMMARY

In view of this, an object of the present disclosure is to provide a method for predicting a dynamic adsorption capacity of VOCs at different concentrations using a static adsorption isotherm. In the present disclosure, the method can predict the dynamic adsorption capacity of different adsorbent materials for VOCs at different concentrations at a same adsorption temperature.

To achieve the above object, the present disclosure provides the following technical solutions:

The present disclosure provides a method for predicting a dynamic adsorption capacity of VOCs at different concentrations using a static adsorption isotherm, the dynamic adsorption capacity including a dynamic saturated adsorption capacity Q_{ds} and a dynamic penetrated adsorption capacity Q_{dp}; where
a process for predicting the dynamic saturated adsorption capacity Q_{ds} includes the following steps:
(1) providing two or more adsorbent materials with significantly different pore sizes, and then testing a static adsorption isotherm of each of the adsorbent materials on VOCs at multiple adsorption temperatures to obtain a static adsorption capacity Qₛ of the VOCs at different pressures;
(2) testing a dynamic adsorption penetration curve of each of the adsorbent materials on the VOCs at multiple concentrations at the multiple adsorption temperatures to obtain the dynamic penetrated adsorption capacity Q_{dp} and the dynamic saturated adsorption capacity Q_{ds} of the VOCs at the multiple concentrations;
(3) calculating a partial pressure corresponding to the VOCs at each of the multiple concentrations in dynamic adsorption based on a saturated vapor pressure of the VOCs at each of the multiple adsorption temperatures and a concentration-partial pressure conversion relationship, and then determining a conversion relationship equation Formula 1 between the dynamic saturated adsorption capacity Q_{ds} and the static adsorption capacities Qₛ at a same partial pressure by statistics of the dynamic saturated adsorption capacity Q_{ds} and the static adsorption capacities Qₛ at the same partial pressure,

$${Q}_{ds} = a \times {Q}_{s} , \qquad Formula 1$$

where
in Formula 1,
Q_{ds} represents the dynamic saturated adsorption capacity, in g/g,
Qₛ represents the static adsorption capacity, in g/g, and
a represents a proportional relationship coefficient between Q_{ds} and Qₛ; and
(4) obtaining the dynamic saturated adsorption capacity Q_{ds} of the VOCs at the same partial pressure in the static adsorption isotherm by combining the static adsorption capacities Qₛ corresponding to each pressure point in the static adsorption isotherm with Formula 1, and then obtaining a curve of the dynamic saturated adsorption capacity Q_{ds} versus the partial pressure according to a change trend of the static adsorption isotherm with a pressure, to obtain a Q_{ds} prediction curve, thereby predicting the dynamic saturated adsorption capacity Q_{ds} of the VOCs at the different concentrations; and
a process for predicting the dynamic penetrated adsorption capacity Q_{dp} includes the following steps:
obtaining a proportional relationship equation Formula 2 between Q_{ds} and Q_{dp} according to a comparison between the dynamic saturated adsorption capacity Q_{ds} and the dynamic penetrated adsorption capacity Q_{dp} at a same concentration and a slope k of the Q_{ds} prediction curve,

$${Q}_{dp} = b \times {Q}_{ds} , \qquad Formula 2$$

where
in Formula 2,
Q_{dp} represents the dynamic penetrated adsorption capacity, in g/g,
Q_{ds} represents the dynamic saturated adsorption capacity, in g/g,
b satisfies an equation b=c×k+d, and b represents a proportional relationship coefficient between Q_{dp} and Q_{ds} at the same partial pressure,
k represents the slope of the Q_{ds} prediction curve,
c and d represent coefficients of the equation b=c×k+d, which are obtained by solving an equation set using the slope k at two different positions on the Q_{ds} prediction curve and a corresponding proportional relationship coefficient b as known numbers; and
obtaining a curve of the dynamic penetrated adsorption capacity Q_{dp} versus the partial pressure by combining the curve of the dynamic saturated adsorption capacity Q_{ds} versus the partial pressure with Formula 2, to obtain a Q_{dp} prediction curve, thereby predicting the dynamic penetrated adsorption capacity Q_{dp} of the VOCs at the different concentrations.

In some embodiments, the process for predicting the dynamic penetrated adsorption capacity Q_{dp} further includes the following steps:
dividing each pressure point on the static adsorption isotherm, the Q_{ds} prediction curve, and the Q_{dp} prediction curve by a saturated vapor pressure at a corresponding temperature to obtain a normalized static adsorption isotherm, a normalized Q_{ds} prediction curve, and a normalized Q_{dp} prediction curve after partial pressure normalization, and then obtaining a general Q_{dp} prediction equation Formula 3 that is not affected by an adsorption temperature difference by combining a slope k₁ of the normalized Q_{ds} prediction curve,

$${Q}_{dp} = {b}_{1} \times {Q}_{ds} , \qquad Formula 3$$

where
in Formula 3,
Q_{dp} represents the dynamic penetrated adsorption capacity, in g/g,
Q_{ds} represents the dynamic saturated adsorption capacity, in g/g,
b₁ satisfies an equation b₁=c₁×k₁+d₁, and b₁ represents a proportional relationship coefficient between Q_{dp} and Q_{ds} at a same relative partial pressure P/P₀, P represents a pressure on the static adsorption isotherm, and P₀ represents a saturated vapor pressure of the VOCs at a specific temperature;
k₁ represents the slope of the normalized Q_{ds} prediction curve relative to the saturated vapor pressure; and
c₁ and d₁ represent coefficients of the equation b₁=c₁×k₁+d₁, which are obtained by solving an equation set using the slope k₁ at two different positions on the normalized Q_{ds} prediction curve and a corresponding proportional relationship coefficient b₁ as known numbers.

In some embodiments, each of the adsorbent materials is independently at least one selected from the group consisting of an activated carbon, a porous silica, and a molecular sieve.

In some embodiments, each of the adsorbent materials independently has an average pore size of 0 nm to 10 nm.

In some embodiments, the significantly different pore sizes indicate that average pore sizes of different adsorbent materials have a difference not less than 2 nm.

In some embodiments, the VOCs are selected from the group consisting of a hydrocarbon organic matter, an oxygen-containing organic matter, a halogen-containing organic matter, a nitrogen-containing organic matter, and a sulfur-containing organic matter.

In some embodiments, in step (1), a number of the multiple adsorption temperatures is not less than 2.

In some embodiments, a concentration number of the VOCs at the multiple concentrations is not less than 2.

The present disclosure provides a method for predicting a dynamic adsorption capacity of VOCs at different concentrations using a static adsorption isotherm. In the present disclosure, based on an objective rule that a partial pressure in the adsorption can have an important influence on the adsorption capacity of VOCs, the static adsorption capacity and the dynamic saturated adsorption capacity at a same partial pressure are compared to find the corresponding relationship between the above two capacities. Based on a change trend of the adsorption capacity in static adsorption with the VOCs pressure, equations and curves of the dynamic saturated adsorption capacity and the dynamic penetrated adsorption capacity in dynamic adsorption changing with the VOCs concentration are obtained, thereby fully reflecting the adsorption performance of the adsorbent material for VOCs, as shown in FIG. 1. The curve of adsorption capacity changing with concentration can be used to obtain the values of dynamic saturated adsorption capacity and penetrated adsorption capacity of VOCs at different concentrations, thereby realizing the prediction of dynamic adsorption capacity. The method helps to improve understanding on the adsorption of gaseous pollutants and build a bridge for the conversion relationship between the dynamic adsorption capacity and the static adsorption capacity of VOCs. As a result, the method can provide a reference for the development of adsorbent materials and technologies suitable for different VOCs waste gases.

### BRIEF DESCRIPTION OF THE DRAWINGS

FIG. 1 shows the equation and curve of the dynamic saturated adsorption capacity and the dynamic penetrated adsorption capacity of VOCs versus the partial pressure obtained the method in an embodiment of the present disclosure based on the static adsorption isotherm;

FIG. 2 shows the pore size distribution curves of each adsorbent material in Example 1;

FIGS. 3A-3D show the static adsorption isotherms of various adsorbent materials on benzene at multiple adsorption temperatures in Example 1;

FIGS. 4A-4D show the dynamic adsorption penetrated curves of various adsorbent materials on benzene at a concentration of 2,000 ppm at multiple adsorption temperatures in Example 1;

FIGS. 5A-5D show the dynamic adsorption penetrated curves of various adsorbent materials on benzene at a concentration of 10% of the saturated vapor pressure (25° C.) at multiple adsorption temperatures in Example 1;

FIGS. 6A-6D show the dynamic adsorption penetrated curves of various adsorbent materials on benzene at a concentration of 50% of the saturated vapor pressure (25° C.) at multiple adsorption temperatures in Example 1;

FIG. 7 shows the fitting results of the linear relationship between the static adsorption capacity and the dynamic saturated adsorption capacity of ACF-2.0, OMC-5.5, and OMC-7.5 at the same partial pressure in Example 1 and the proportional relationship formula between the two capacities;

FIGS. 8A-8C show the prediction curve of Q_{ds} versus the partial pressure obtained according to the correlation relationship between Qₛ and Q_{ds} at the same partial pressure in Example 1 and the comparison of the obtained values with the measured values;

FIG. 9 shows the adsorption isotherm of OMC-5.5 at 35° C. and the curve of the dynamic saturated adsorption capacity Q_{ds} versus the partial pressure in Example 1, as well as the influence of the slope k of the curve on the difference between Q_{ds} and Q_{dp};

FIGS. 10A-10C show the prediction equation and curve of Q_{dp} versus the partial pressure at each temperature obtained according to the correlation relationship between Q_{ds} and Q_{dp} at 25° C., 35° C., and 45° C. in Example 1;

FIGS. 11A-11D show the adsorption isotherms at relative partial pressures normalized (P/P₀) to the saturated vapor pressure (P₀) at respective adsorption temperatures in Example 2;

FIG. 12 shows the normalized (P/P₀) adsorption isotherm and the curve of Q_{ds} versus the relative partial pressure in Example 2, as well as the influence of the slope of the curve on the difference between Q_{ds} and Q_{dp};

FIGS. 13A-13D show the general Q_{dp} prediction equation applicable to different adsorption temperatures and the prediction curve of Q_{dp} versus the partial pressure at different adsorption temperatures obtained according to the normalized (P/P₀) correlation relationship between Q_{ds} and Q_{dp} in Example 2; and

FIGS. 14A-14B show the prediction curves of Q_{ds} and Q_{dp} obtained using Formula 3 in Example 2, and the comparison of the obtained Q_{ds} and Q_{dp} values with the measured Q_{ds} and Q_{dp} values.

### DETAILED DESCRIPTION OF THE EMBODIMENTS

The present disclosure provides a method for predicting a dynamic adsorption capacity of VOCs at different concentrations using a static adsorption isotherm, the dynamic adsorption capacity including a dynamic saturated adsorption capacity Q_{ds} and a dynamic penetrated adsorption capacity Q_{dp}; where a process for predicting the dynamic saturated adsorption capacity Q_{ds} includes the following steps:
(1) providing two or more adsorbent materials with significantly different pore sizes, and then testing a static adsorption isotherm of each of the adsorbent materials on VOCs at multiple adsorption temperatures to obtain a static adsorption capacity Qₛ of the VOCs at different pressures;
(2) testing a dynamic adsorption penetration curve of each of the adsorbent materials on the VOCs at multiple concentrations at the multiple adsorption temperatures to obtain the dynamic penetrated adsorption capacity Q_{dp} and the dynamic saturated adsorption capacity Q_{ds} of the VOCs at a specific concentration;
(3) calculating a partial pressure corresponding to the VOCs at each of the multiple concentrations in dynamic adsorption based on a saturated vapor pressure of the VOCs at each of the multiple adsorption temperatures and a concentration-partial pressure conversion relationship, and then determining a conversion relationship equation Formula 1 between the dynamic saturated adsorption capacity Q_{ds} and the static adsorption capacities Qₛ at a same partial pressure by statistics of the dynamic saturated adsorption capacity Q_{ds} and the static adsorption capacities Qₛ at the same partial pressure;

$${Q}_{ds} = a \times {Q}_{s} , \qquad Formula 1$$

where
in Formula 1,
Q_{ds} represents the dynamic saturated adsorption capacity, in g/g;
Qₛ represents the static adsorption capacity, in g/g; and
a represents a proportional relationship coefficient between Q_{ds} and Qₛ; and
(4) obtaining the dynamic saturated adsorption capacity Q_{ds} of the VOCs at the same partial pressure in the static adsorption isotherm by combining the static adsorption capacity Qₛ corresponding to each pressure point in the static adsorption isotherm with Formula 1, and then obtaining a curve of the dynamic saturated adsorption capacity Q_{ds} versus the partial pressure according to a change trend of the static adsorption isotherm with a pressure, to obtain a Q_{ds} prediction curve, thereby predicting the dynamic saturated adsorption capacity Q_{ds} of the VOCs at the different concentrations; and
a process for predicting the dynamic penetrated adsorption capacity Q_{dp} includes the following steps:
obtaining a proportional relationship equation Formula 2 between Q_{ds} and Q_{dp} according to a comparison between the dynamic saturated adsorption capacity Q_{ds} and the dynamic penetrated adsorption capacity Q_{dp} at a same concentration and a slope k of the Q_{ds} prediction curve;

$${Q}_{dp} = b \times {Q}_{ds} , \qquad Formula 2$$

where
in Formula 2,
Q_{dp} represents the dynamic penetrated adsorption capacity, in g/g;
Q_{ds} represents the dynamic saturated adsorption capacity, in g/g;
b satisfies an equation b=c×k+d, and b represents a proportional relationship coefficient between Q_{dp} and Q_{ds} at the same partial pressure;
k represents the slope of the Q_{ds} prediction curve;
c and d represent coefficients of the equation b=c×k+d, which are obtained by solving an equation set using the slope k at two different positions on the Q_{ds} prediction curve and a corresponding proportional relationship coefficient b as known numbers; and
obtaining a curve of the dynamic penetrated adsorption capacity Q_{dp} versus the partial pressure by combining the curve of the dynamic saturated adsorption capacity Q_{ds} versus the partial pressure with Formula 2, to obtain a Q_{dp} prediction curve, thereby predicting the dynamic penetrated adsorption capacity Q_{dp} of the VOCs at the different concentrations.

In the present disclosure, a process for predicting the dynamic saturated adsorption capacity Q_{ds} includes the following steps:

In the present disclosure, two or more adsorbent materials with significantly different pore sizes are provided, and then a static adsorption isotherm of each of the adsorbent materials on VOCs at multiple adsorption temperatures is tested to obtain a static adsorption capacity Qₛ of the VOCs at different pressures. In some embodiments of the present disclosure, each of the adsorbent materials is independently at least one selected from the group consisting of an activated carbon, a porous silica, and a molecular sieve. In some embodiments of the present disclosure, each of the adsorbent materials independently has an average pore size of 0 nm to 10 nm, and a pore size distribution of each adsorbent material is within a range of micropores and small mesopores. In some embodiments of the present disclosure, the significantly different pore sizes indicate that average pore sizes of different adsorbent materials have a difference not less than 2 nm.

As a specific embodiment of the present disclosure, the adsorbent materials include ACF-2.0, OMC-5.5, or/and OMC-7.5, the ACF-2.0 has an average pore size of 2.0 nm, the OMC-5.5 has an average pore size of 5.5 nm, and the OMC-7.5 has an average pore size of 7.5 nm.

In some embodiments the present disclosure, the VOCs are selected from the group consisting of a hydrocarbon organic matter, an oxygen-containing organic matter, a halogen-containing organic matter, a nitrogen-containing organic matter, and a sulfur-containing organic matter. In some embodiments of the present disclosure, the hydrocarbon organic matter is selected from the group consisting of an alkane, an alkene, an alkyne, and an aromatic hydrocarbon; the oxygen-containing organic matter is selected from the group consisting of an aldehyde organic matter, a ketone organic matter, an alcohol organic matter, and an ester organic matter. As a specific embodiment, the VOCs are benzene.

In some embodiments of the present disclosure, a number of the multiple adsorption temperatures is not less than 2. As a specific embodiment, the multiple adsorption temperatures include 25° C., 35° C., and 45° C.

In the present disclosure, the static adsorption isotherm is a curve showing changes of the static adsorption capacity Qₛ with a pressure.

In the present disclosure, a dynamic adsorption penetration curve of each of the adsorbent materials on the VOCs at multiple concentrations at the multiple adsorption temperatures is tested to obtain the dynamic penetrated adsorption capacity Q_{dp} and the dynamic saturated adsorption capacity Q_{ds} of the VOCs at a specific concentration. In some embodiments of the present disclosure, the dynamic penetrated adsorption capacity Q_{dp} and the dynamic saturated adsorption capacity Q_{ds} are calculated by combining a gas flow rate, a concentration of VOCs, an amount of each adsorbent material, a penetration time, and a saturation time.

In some embodiments of the present disclosure, a concentration number of the VOCs at the multiple concentrations is not less than 2. As a specific embodiment, the multiple concentrations of the VOCs include 2,000 ppm, 10% saturated vapor pressure, and 50% saturated vapor pressure.

In some embodiments of the present disclosure, the dynamic saturated adsorption capacity Q_{ds} of the VOCs is calculated according to Formula 4:

$${Q}_{ds} = \frac{F \times M}{{V}_{m} \times m} ( {c}_{0} {t}_{ds} - {\int }_{0}^{{t}_{ds}} {c}_{i} dt ) , \qquad Formula 4$$

where
in Formula 4,
Q_{ds} represents the dynamic saturated adsorption capacity, in g/g;
F represents a total gas flow rate, in L/min;
M represents a molar mass of an adsorbate, in g/mol;
Vₘ represents a molar volume of a gas, satisfying Vₘ=22.4 L/mol;
m represents a net weight of an adsorbent, in g;
c₀ represents an initial VOCs concentration at an inlet of an adsorbent bed, in ppm;
cᵢ represents a VOCs concentration at an outlet of the adsorbent bed, in ppm, and when adsorption saturation is satisfied, cᵢ=c₀;
t represents a adsorption time, in min;
t_{ds} represents the saturation time, which is a time taken for when an outlet concentration of the adsorbent bed is the same as an inlet concentration, in min.

In some embodiments of the present disclosure, the dynamic penetrated adsorption capacity Q_{dp} of the VOCs is calculated according to Formula 5:

$${Q}_{dp} = \frac{F \times M}{{V}_{m} \times m} ( {c}_{0} {t}_{dp} - {\int }_{0}^{{t}_{dp}} {c}_{i} dt ) , \qquad Formula 5$$

where
in Formula 5,
the meanings of F, M, Vₘ, m, c₀, and t are the same as those in Formula 4;
Q_{dp} represents the dynamic penetrated adsorption capacity, in g/g;
t_{dp} represents the penetration time, which is a time taken for the VOCs concentration at the outlet of the adsorbent bed to reach 5% of the inlet concentration, in min;
cᵢ represents the VOCs concentration at the outlet of the adsorbent bed, in ppm, and when adsorption penetration is satisfied, cᵢ=5%×c₀.

In the present disclosure, a partial pressure is calculated corresponding to the VOCs at each of the multiple concentrations in dynamic adsorption based on a saturated vapor pressure of the VOCs at each of the multiple adsorption temperatures and a concentration-partial pressure conversion relationship, and then a conversion relationship between the dynamic saturated adsorption capacity Q_{ds} and the static adsorption capacities Qₛ at a same partial pressure is determined by statistics of the dynamic saturated adsorption capacity Q_{ds} and the static adsorption capacities Qₛ at the same partial pressure, namely Formula 1;

$${Q}_{ds} = a \times {Q}_{s} , \qquad Formula 1$$

where
in Formula 1,
Q_{ds} represents the dynamic saturated adsorption capacity, in g/g;
Qₛ represents the static adsorption capacity, in g/g; and
a represents a proportional relationship coefficient between Q_{ds} and Qₛ.

In the present disclosure, a concentration of dynamic adsorption and a pressure of static adsorption essentially reflect an amount of VOCs molecules in a unit space, and both can be expressed by partial pressure. The pressure of static adsorption of the VOCs is in mbar, and the concentration of dynamic adsorption of the VOCs is in ppm or percentage of saturated vapor pressure, both can be uniformly converted to mbar, that is, partial pressure. For specific VOCs, each temperature corresponds to a specific saturated vapor pressure constant.

In the present disclosure, the dynamic saturated adsorption capacity Q_{ds} of the VOCs at the same partial pressure in the static adsorption isotherm is obtained by combining the static adsorption capacities Qₛ corresponding to each pressure point in the static adsorption isotherm with Formula 1, and then a curve of the dynamic saturated adsorption capacity Q_{ds} versus the partial pressure is obtained according to a change trend of the static adsorption isotherm with a pressure, to obtain a Q_{ds} prediction curve, thereby predicting the dynamic saturated adsorption capacity Q_{ds} of the VOCs at the different concentrations.

In the present disclosure, a process for predicting the dynamic penetrated adsorption capacity Q_{dp} includes the following steps:

In the present disclosure, a proportional relationship equation between Q_{ds} and Q_{dp} is obtained according to a comparison between the dynamic saturated adsorption capacity Q_{ds} and the dynamic penetrated adsorption capacity Q_{dp} at a same concentration and a slope k of the Q_{ds} prediction curve, namely Formula 2;

$${Q}_{dp} = b \times {Q}_{ds} , \qquad Formula 2$$

where
in Formula 2,
Q_{dp} represents the dynamic penetrated adsorption capacity, in g/g;
Q_{ds} represents the dynamic saturated adsorption capacity, in g/g;
b satisfies an equation b=c×k+d, and b represents a proportional relationship coefficient between Q_{dp} and Q_{ds} at the same partial pressure;
k represents the slope of the Q_{ds} prediction curve;
c and d represent coefficients of the equation b=c×k+d, which are obtained by solving an equation set using the slope k at two different positions on the Q_{ds} prediction curve and a corresponding proportional relationship coefficient b as known numbers; and
a curve of the dynamic penetrated adsorption capacity Q_{dp} versus the partial pressure is obtained by combining the curve of the dynamic saturated adsorption capacity Q_{ds} versus the partial pressure with Formula 2, i.e., a Q_{dp} prediction curve, thereby predicting the dynamic penetrated adsorption capacity Q_{dp} of the VOCs at the different concentrations. Specifically, based on the comparison of the dynamic saturated adsorption capacity Q_{ds} and the dynamic penetrated adsorption capacity Q_{dp} at the same concentration, and combined with the characteristics of dynamic penetration curves of different materials, the proportional relationship between the above two is obtained, that is, Formula 2; combined with the curve of the dynamic saturated adsorption capacity Q_{ds} versus the partial pressure, the curve of the dynamic penetrated adsorption capacity Q_{dp} versus the partial pressure is obtained, such that the dynamic penetrated adsorption capacity Q_{dp} can be predicted at the different concentrations.

In some embodiments of the present disclosure, each pressure point on the static adsorption isotherm, the Q_{ds} prediction curve, and the Q_{dp} prediction curve is divided by a saturated vapor pressure at a corresponding temperature to obtain a normalized static adsorption isotherm, a normalized Q_{ds} prediction curve, and a normalized Q_{dp} prediction curve after partial pressure normalization, and then a general Q_{dp} prediction equation that is applicable to different adsorption temperatures and not affected by an adsorption temperature difference is obtained by combining a slope k₁ of the normalized Q_{ds} prediction curve, namely Formula 3;

$${Q}_{dp} = {b}_{1} \times {Q}_{ds} , \qquad Formula 3$$

where
in Formula 3,
Q_{dp} represents the dynamic penetrated adsorption capacity, in g/g;
Q_{ds} represents the dynamic saturated adsorption capacity, in g/g;
b₁ satisfies an equation b₁=c₁×k₁+d₁, and b₁ represents a proportional relationship coefficient between Q_{dp} and Q_{ds} at a same relative partial pressure P/P₀, P represents a pressure on the static adsorption isotherm, and P₀ represents a saturated vapor pressure of the VOCs at a specific temperature;
k₁ represents the slope of the normalized Q_{ds} prediction curve relative to the saturated vapor pressure; and
c₁ and d₁ represent coefficients of the equation b₁=c₁×k₁+d₁, which are obtained by solving an equation set using the slope k₁ at two different positions on the Q_{ds} prediction curve and a corresponding proportional relationship coefficient b₁ as known numbers.

In some embodiments of the present disclosure, Q_{ds} prediction curves at different adsorption temperatures are normalized relative to the saturated vapor pressure at each temperature to obtain a curve of Q_{dp} versus concentration at the different temperatures using Formula 3, thereby predicting the dynamic penetrated adsorption capacity Q_{dp} at the different temperatures and concentrations.

A concentration of the VOCs in a dynamic adsorption test is matched with a pressure of the VOCs in a static adsorption test reveals a correlation relationship between the dynamic adsorption capacity and the static adsorption capacity at a same partial pressure. A change of the adsorption capacity due to different partial pressures in the static adsorption isotherm reveals a law of how the adsorption capacity of adsorbent materials versus VOCs concentration during dynamic adsorption, and an equation and a curve for predicting the adsorption capacity of the dynamic saturated adsorption capacity and the dynamic penetrated adsorption capacity versus the partial pressure are obtained. Furthermore, by normalizing a Q_{ds} prediction curve relative to a saturated vapor pressure and combining the influence of the slope k, a general Q_{dp} prediction equation that is not affected by an adsorption temperature difference is obtained to effectively overcome the influence of the adsorption temperature difference, thus achieving prediction of the dynamic saturated adsorption capacity and the dynamic penetrated adsorption capacity of the VOCs at the different temperatures and different concentrations.

The method for predicting the dynamic adsorption capacity of VOCs at different concentrations using the static adsorption isotherm provided by the present disclosure is described in detail below with reference to the examples, but these examples may not be understood as a limitation to the scope of the present disclosure.

### EXAMPLE 1

(1) Two or more adsorbent materials with significantly different pore sizes were provided, and then a static adsorption isotherm of each of the adsorbent materials on VOCs at multiple adsorption temperatures was tested to obtain a static adsorption capacity Qₛ of the VOCs at different pressures.

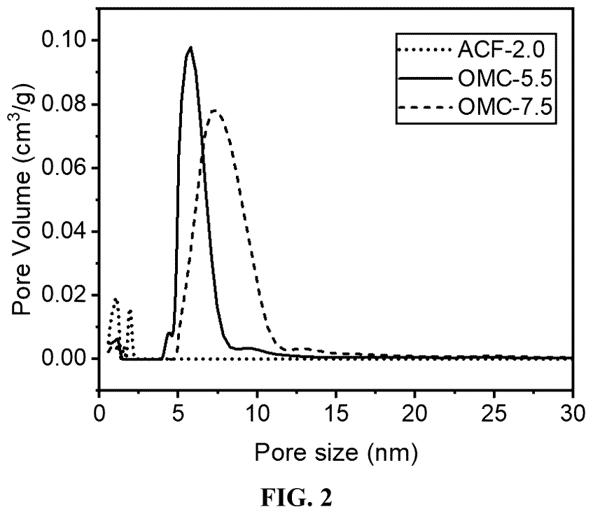

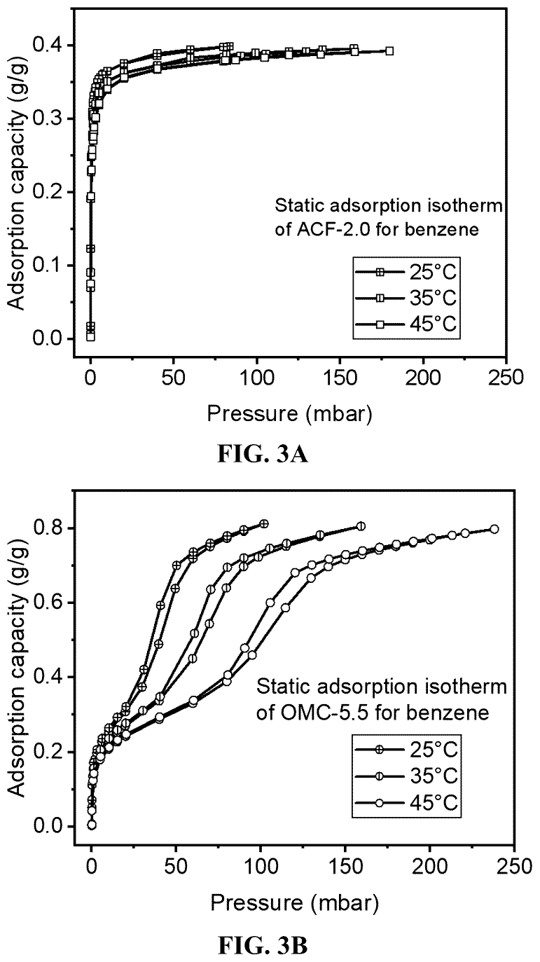

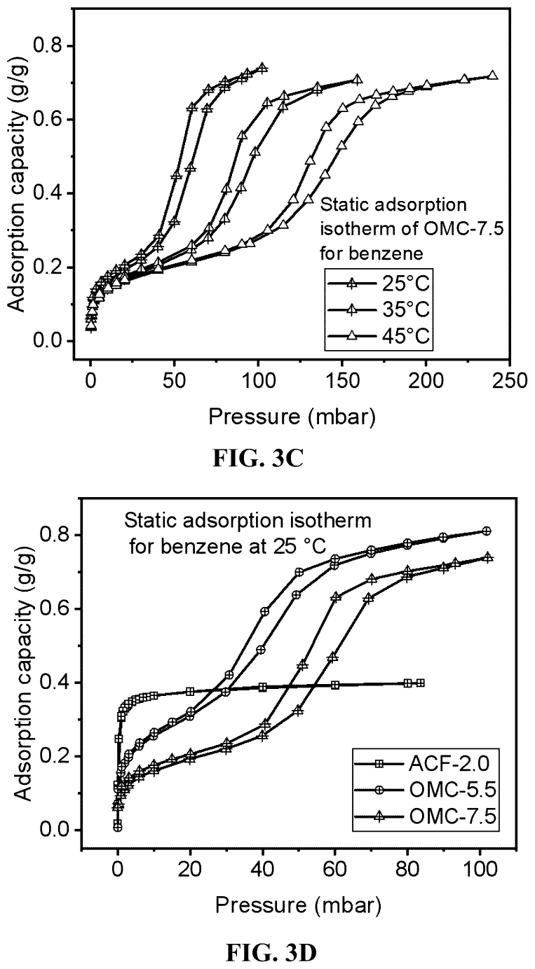

ACF-2.0, OMC-5.5, and OMC-7.5 carbon materials with average pore sizes of 2.0 nm, 5.5 nm, and 7.5 nm, respectively, were used as adsorbents (FIG. 2) to test their static adsorption isotherms for benzene at 25° C., 35° C., and 45° C., respectively (FIGS. 3A-3D).

The static adsorption isotherm of benzene adsorption on activated carbon fibers with micropores is mainly a typical type I adsorption isotherm, showing typical characteristics that the adsorption capacity increases rapidly and approaches saturation under a relatively low pressure, and the increase in pressure has little effect on the adsorption capacity. This is mainly because the pore structure of ACF-2.0 is mainly micropores below 2 nm. The superposition of an adsorption potential of adjacent pore walls in micropores with a size similar to that of VOCs molecules leads to an enhanced adsorption force, resulting in micropore filling manifested in the adsorption isotherm as a type I adsorption isotherm.

The adsorption of benzene on mesoporous OMC materials is a typical type IV adsorption isotherm, which roughly includes three stages. In the initial stage of adsorption, the adsorption capacity increases slowly with the increase of vapor pressure, which should be attributed to the process in which benzene molecules gradually form a monolayer adsorption on the pore surface of OMCs. In the middle stage of adsorption, the adsorption capacity of benzene rises rapidly on the isotherm, which is caused by the capillary condensation occurring in the concentrated pores. In the third stage of adsorption, the isotherm reaches a plateau and the adsorption capacity increases only slightly, which is mainly caused by the rearrangement of benzene molecules adsorbed in the pores of OMCs in a volume-filling manner. In the middle stage of the type IV adsorption isotherm, the filling adsorption caused by capillary condensation has a large contribution to the adsorption capacity, accounting for more than 50% of a total adsorption capacity.

(2) A dynamic adsorption penetration curve of each of the adsorbent materials on the VOCs at multiple concentrations at the multiple adsorption temperatures was tested to obtain the dynamic penetrated adsorption capacity Q_{dp} and the dynamic saturated adsorption capacity Q_{ds} of the VOCs at a specific concentration combined with a gas flow rate, a VOCs concentration, an amount of each adsorbent material, a penetration time, and a saturation time.

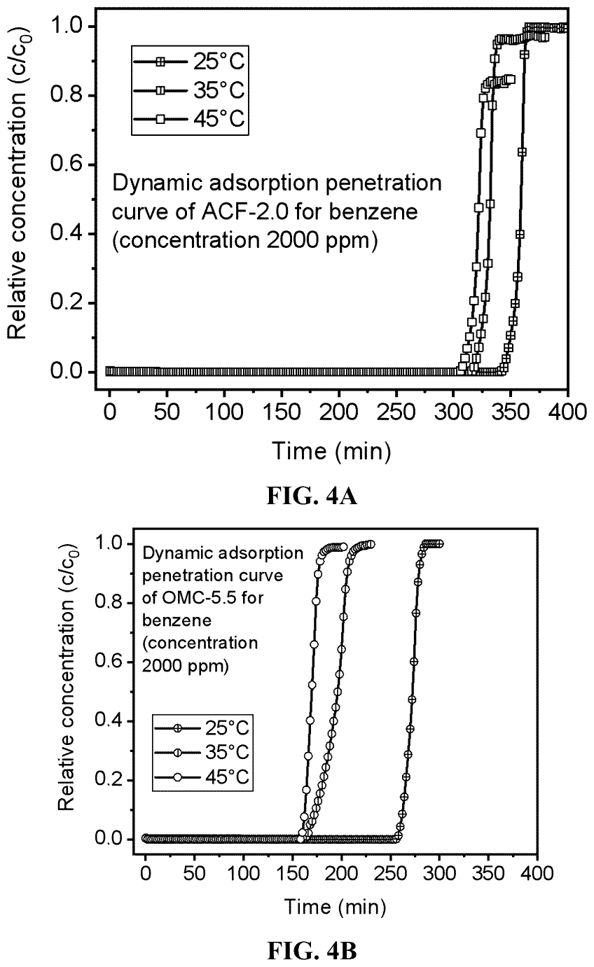

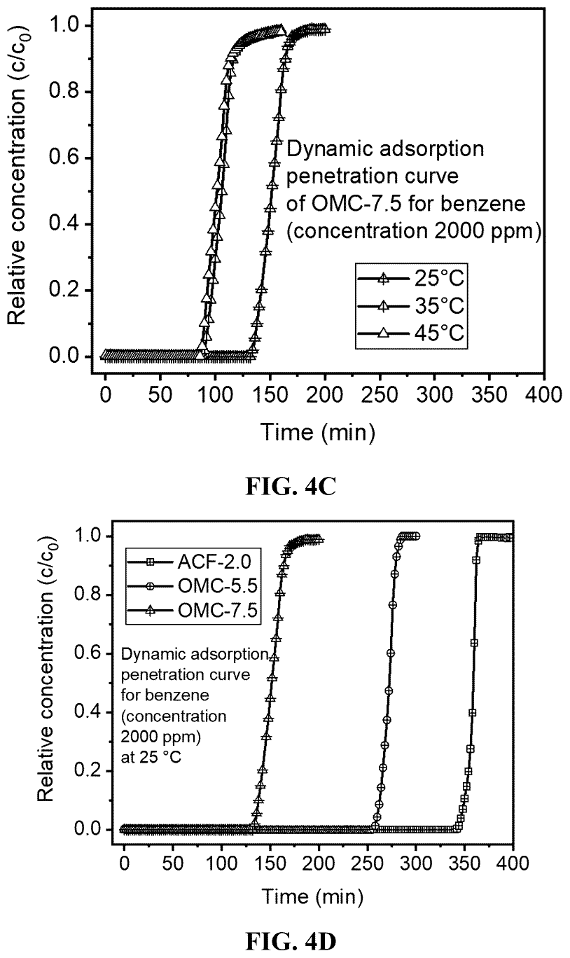

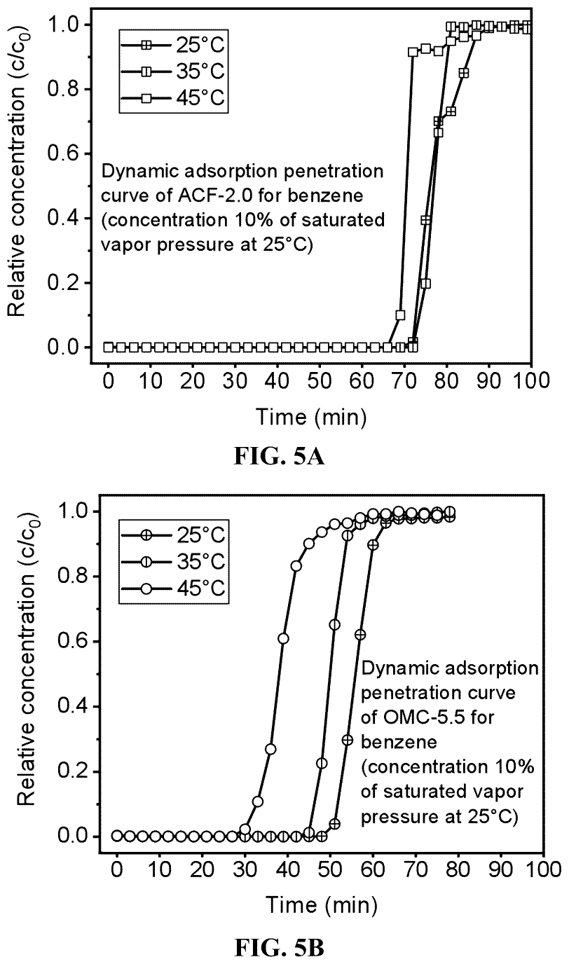

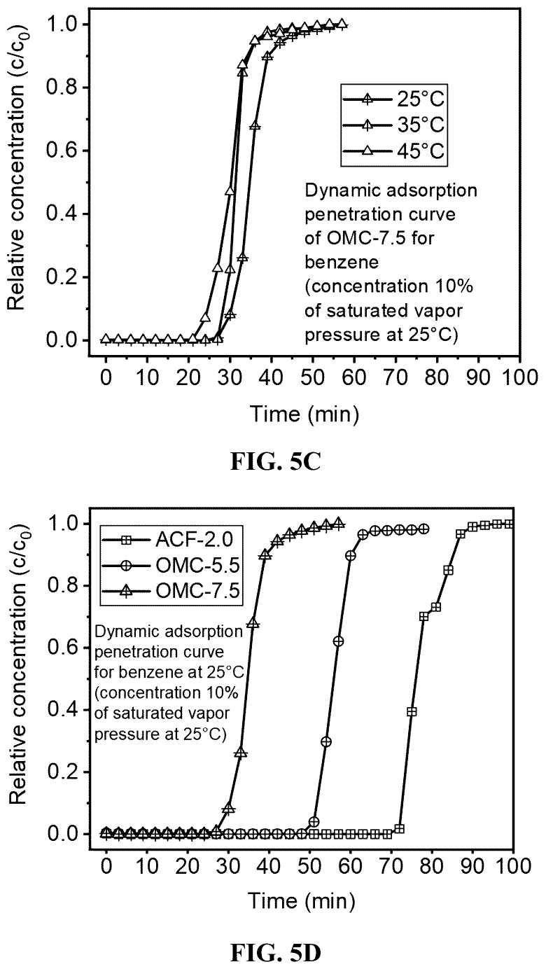

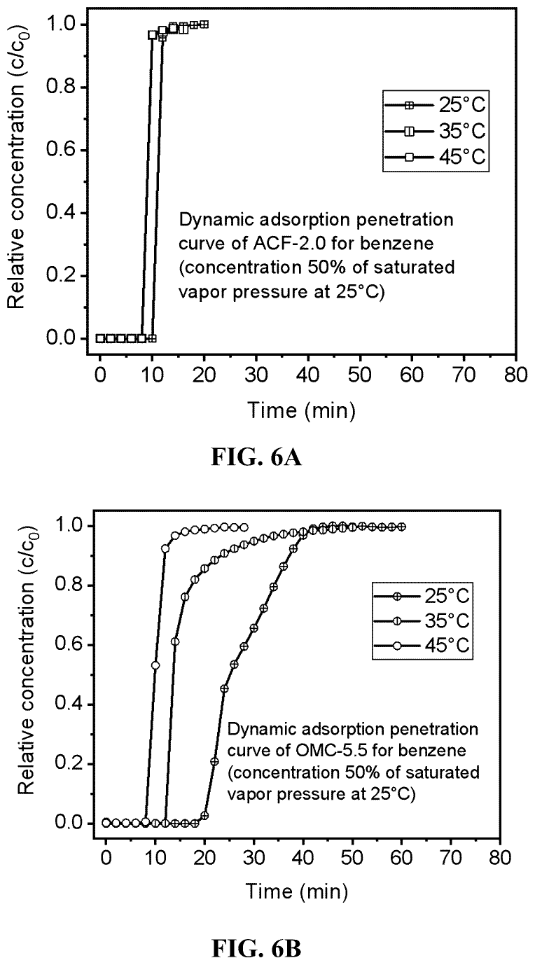

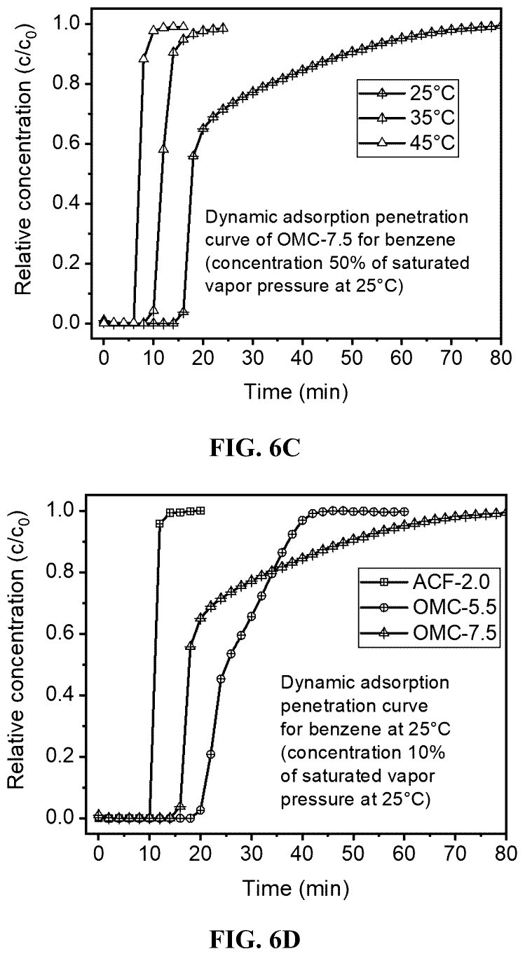

Dynamic adsorption penetration curves of ACF-2.0, OMC-5.5, and OMC-7.5 at 25° C., 35° C., and 45° C. for benzene with concentrations of 2,000 ppm, 10% saturated vapor pressure (25° C.), and 50% saturated vapor pressure (25° C.) were tested (FIGS. 4A-4D, FIGS. 5A-5D, FIGS. 6A-6D, respectively). The dynamic saturated adsorption capacity and the dynamic penetrated adsorption capacity of benzene at a specific concentration were calculated based on a penetration curve combined with the gas flow rate, a benzene concentration, the amount of each adsorbent material, the penetration time, and the saturation time.

The dynamic saturated adsorption capacity of each of the adsorbent materials was calculated by Formula 4:

$${Q}_{ds} = \frac{F \times M}{{V}_{m} \times m} ( {c}_{0} {t}_{ds} - {{\int }^{{t}_{ds}}}_{0} {c}_{i} dt ) , \qquad Formula 4$$

where
in Formula 4,
Q_{ds} represented the dynamic saturated adsorption capacity, in g/g;
F represented a total gas flow rate, in L/min;
M represented a molar mass of an adsorbate, in g/mol;
Vₘ represented a molar volume of a gas, satisfying Vₘ=22.4 L/mol;
m represented a net weight of an adsorbent, in g;
c₀ represented an initial VOCs concentration at an inlet of an adsorbent bed, in ppm;
cᵢ represented a VOCs concentration at an outlet of the adsorbent bed, in ppm, and when adsorption saturation was satisfied, cᵢ=c₀;
t represented an adsorption time, in min;
t_{ds} represented the saturation time, which was a time taken for when an outlet concentration of the adsorbent bed was the same as an inlet concentration.

The dynamic penetrated adsorption capacity of each of the adsorbent materials was calculated by Formula 5:

$${Q}_{dp} = \frac{F \times M}{{V}_{m} \times m} ( {c}_{0} {t}_{dp} - {{\int }^{{t}_{dp}}}_{0} {c}_{i} dt ) , \qquad Formula 5$$

where
in Formula 5,
the meanings of F, M, Vₘ, m, c₀, and t were the same as those in Formula 4;
Q_{dp} represented the dynamic penetrated adsorption capacity, in g/g;
t_{dp} represented the penetration time, which was a time taken for the VOCs concentration at the outlet of the adsorbent bed to reach 5% of the inlet concentration, in min;
cᵢ represented the VOCs concentration at the outlet of the adsorbent bed, in ppm, and when adsorption penetration was satisfied, cᵢ=5%×C₀.

In this example, the adsorbate used was benzene, with a total flow rate of F=0.02 L/min; gas inlet concentrations were 2,000 ppm, 10% benzene saturated vapor pressure at 25° C. (i.e. 10%×127.61 mbar÷1,000 mbar=12.8×10³ ppm), and 50% benzene saturated vapor pressure at 25° C. (i.e. 50%×127.61 mbar÷1,000 mbar=63.8×10³ ppm); a molar mass of benzene was M=78 g/mol; an amount of adsorbent used was m=0.2 g.

It is seen from the adsorption penetration curve that with the increase of adsorption temperature, the saturated and penetrated adsorption capacities gradually decrease, which is reflected in shorter penetration time and saturation time on the penetration curve, and there are certain differences in the sensitivity of different adsorbent materials to temperature at different concentrations. When the same adsorbents adsorb multiple concentrations of benzene at the same temperature, the adsorption penetration time and saturation time gradually shorten as the benzene concentration increases, but the adsorption capacity is usually greater due to a higher concentration. Since micropores have a relatively strong adsorption capacity, filling-type adsorption could be realized at relatively low concentrations to achieve a relatively high pore volume utilization rate, and therefore is less affected by concentration changes. The pore volume utilization rate of larger mesopores is greatly affected by the VOCs concentration. The adsorption capacity is poor at low concentrations. A high adsorption utilization rate could be maintained only when the concentration exceeds a certain value and most of the pore volume could be filled with adsorption. The results of the dynamic saturated adsorption capacity versus concentration are similar to the trend of the static adsorption capacity versus pressure.

(3) A partial pressure corresponding to the VOCs at each of the multiple concentrations in dynamic adsorption was calculated based on a saturated vapor pressure of the VOCs at a specific adsorption temperature and a concentration-partial pressure conversion relationship, and then a conversion relationship between the dynamic saturated adsorption capacity Q_{ds} and the static adsorption capacities Qₛ at a same partial pressure was determined by statistics of the dynamic saturated adsorption capacity Q_{ds} and the static adsorption capacities Qₛ at the same partial pressure, namely Formula 1;

$${Q}_{ds} = a \times {Q}_{s} , \qquad Formula 1$$

where
in Formula 1,
Q_{ds} represented the dynamic saturated adsorption capacity, in g/g;
Qₛ represented the static adsorption capacity, in g/g; and
a represented a proportional relationship coefficient between Q_{ds} and Qₛ.

A saturated vapor pressure of benzene at 25° C. was 127.61 mbar. Calculated partial pressures corresponding to concentrations of 2,000 ppm (ppm represented parts per million), 10% saturated vapor pressure (25° C.), and 50% saturated vapor pressure (25° C.) were 2 mbar, 12.8 mbar, and 63.8 mbar, respectively.

A calculation process was as follows:

$$2000 ppm \times 1000 mbar = 2 mbar ;$$

$$10 \% \times {P}_{0} = 12.8 mbar ;$$

$$50 \% \times {P}_{0} = 63.8 mbar ;$$

where
P₀ represented a saturated vapor pressure of the VOCs at a specific temperature, and the saturated vapor pressure of benzene at 25° C. was 127.61 mbar.

Dynamic saturated adsorption capacities of ACF-2.0, OMC-5.5, and OMC-7.5 at partial pressures of 2 mbar, 12.8 mbar, and 63.8 mbar, respectively, as well as static adsorption capacities at the same partial pressures are shown in Table 1. The comparison shows that under the same conditions, the dynamic saturated adsorption capacity is generally low than the static adsorption capacity. This is because under dynamic adsorption conditions, benzene as the adsorbate is continuously transported to the adsorbent bed in a N₂ gas flow. Before and during dynamic adsorption, pores of the adsorbent are not vacuum, but occupied by a certain amount of gas (such as nitrogen, air). Compared with static adsorption, dynamic adsorption of the VOCs on the adsorbent materials also involves the diffusion and replacement of VOCs molecules and other gases in the pores. Due to incomplete replacement, some non-VOCs gases (such as nitrogen, air) might occupy the pores with VOCs adsorption capacity, resulting in a loss of adsorption capacity. Therefore, the dynamic saturated adsorption capacity is generally slightly lower than the static adsorption capacity.

TABLE 1
Static adsorption capacity (Qₛ) and dynamic saturated adsorption
capacity (Q_{ds}) of benzene for each adsorbent material at
partial pressures of 2 mbar, 12.8 mbar, and 63.8 mbar

| | | 25° C. | | 35° C. | | 45° C. | |
|---|---|---|---|---|---|---|---|
| Adsorbent | P | Qₛ | Q_{ds} | Qₛ | Q_{ds} | Qₛ | Q_{ds} |
| material | (mbar) | (g/g) | (g/g) | (g/g) | (g/g) | (g/g) | (g/g) |
| ACF-2.0 | 2.0 | 0.330 | 0.249 | 0.306 | 0.230 | 0.284 | 2.224 |
| | 12.8 | 0.367 | 0.345 | 0.3530 | 0.3420 | 0.3430 | 0.3170 |
| | 63.8 | 0.393 | 0.247 | 0.379 | 0.204 | 0.374 | 0.202 |
| OMC-5.5 | 2.0 | 0.183 | 0.189 | 0.1560 | 0.1340 | 0.1410 | 0.1180 |
| | 12.70 | 0.271 | 0.250 | 0.2430 | 0.2240 | 0.2180 | 0.1720 |
| | 63.8 | 0.729 | 0.613 | 0.486 | 0.358 | 0.341 | 0.228 |
| OMC-7.5 | 2.0 | 0.111 | 0.106 | 0.0920 | 0.0740 | 0.0920 | 0.0720 |
| | 12.8 | 0.170 | 0.156 | 0.1510 | 0.1410 | 0.1460 | 0.1330 |
| | 63.8 | 0.535 | 0.560 | 0.259 | 0.271 | 0.221 | 0.163 |

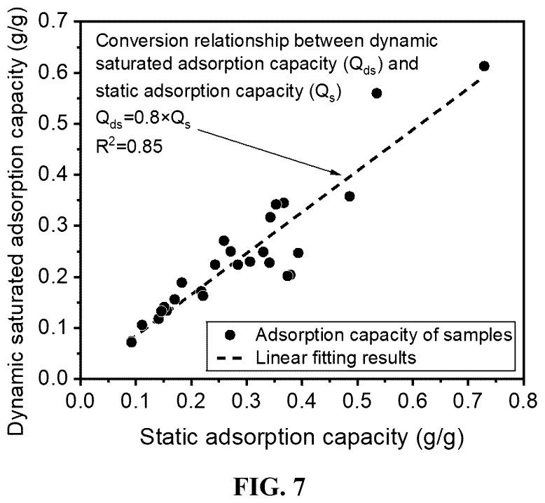

Based on the above static adsorption experiments and dynamic adsorption experiments, the static adsorption capacity and the dynamic saturated adsorption capacity at the same partial pressure were obtained, as shown in Table 1. By linear fitting on the static adsorption capacity (Qₛ) and the dynamic saturated adsorption capacity (Q_{ds}) at the same partial pressure, it was found that the two adsorption capacity values could be well fitted, and a linear relationship equation Q_{ds}=0.8×Qₛ was obtained, that is, the dynamic saturated adsorption capacity at the same partial pressure is approximately 0.8 times the static adsorption capacity at a corresponding partial pressure, as shown in FIG. 7.

(4) A dynamic saturated adsorption capacity Q_{ds} of the VOCs at the same partial pressure in the static adsorption isotherm was obtained by combining the static adsorption capacity Qₛ corresponding to each pressure point in the static adsorption isotherm with Formula 1, and then a curve of the dynamic saturated adsorption capacity Q_{ds} versus the partial pressure was obtained according to a change trend of the static adsorption isotherm with a pressure, to obtain a Q_{ds} prediction curve, thereby predicting the dynamic saturated adsorption capacity Q_{ds} of the VOCs at the different concentrations.

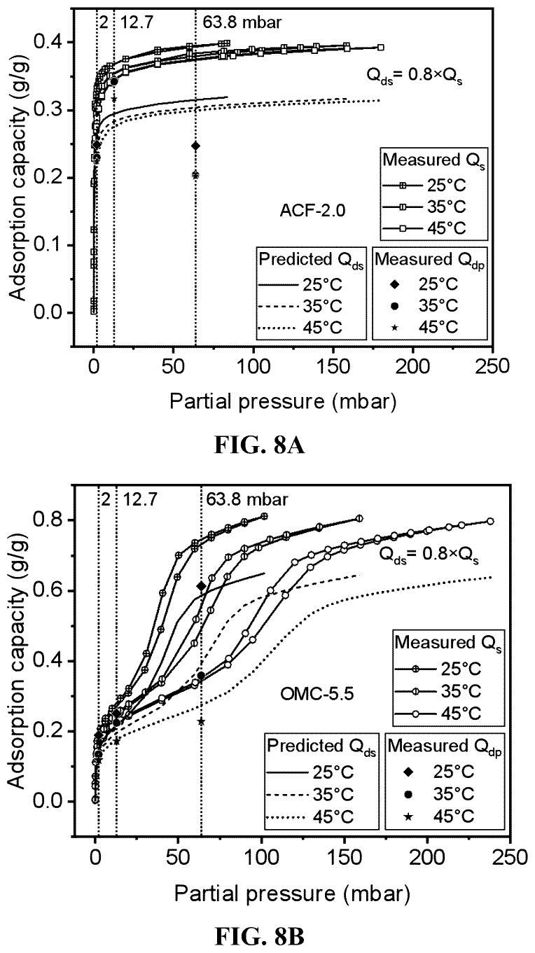

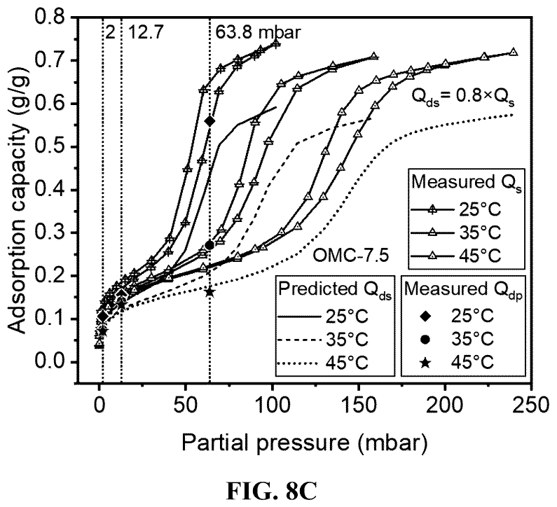

A empirical formula Q_{ds}=0.8×Qₛ was combined with the static adsorption isotherm to obtain a predicted value of the dynamic saturated adsorption capacity at the same partial pressure and a law of its change with partial pressure, as shown in FIGS. 8A-8C. In the adsorption, based on an objective rule that the partial pressure in the adsorption could have an important influence on the adsorption capacity of the VOCs, the static adsorption capacity and the dynamic saturated adsorption capacity at a same partial pressure are compared to find a corresponding relationship between the above two capacities. A changing trend of the adsorption capacity of adsorbent materials for VOCs in static adsorption with a increase of VOCs pressure could be explored, and a curve of the dynamic saturated adsorption capacity versus VOCs concentration in dynamic adsorption could fully reflect the adsorption performance of the adsorbent materials for the VOCs.

(5) Based on a comparison of the dynamic saturated adsorption capacity Q_{ds} and the dynamic penetrated adsorption capacity Q_{dp} at a same concentration, and combined with an influence of a slope k of the Q_{ds} prediction curve on a difference between Q_{ds} and Q_{dp}, a proportional relationship equation between the two capacities was obtained; and combined with the curve of the dynamic saturated adsorption capacity Q_{ds} versus the partial pressure, a curve of the dynamic penetrated adsorption capacity Q_{dp} versus concentration was obtained using Formula 2, thereby predicting the dynamic penetrated adsorption capacity Q_{dp} at the different concentrations.

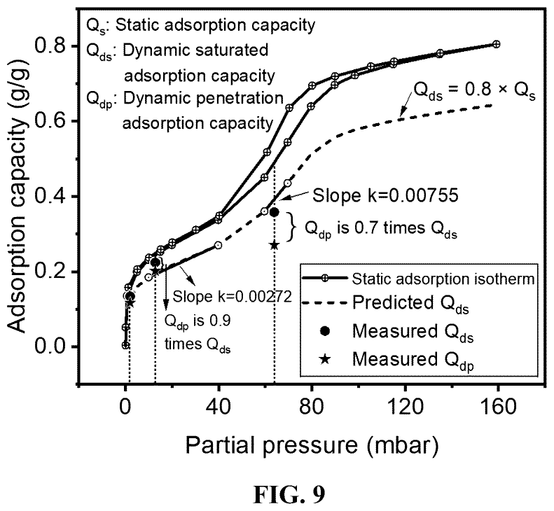

The dynamic penetrated adsorption capacity and the dynamic saturated adsorption capacity of each adsorbent are shown in Table 2, and the difference between the two capacities is affected by factors such as a pore structure of materials, the adsorption temperature, and the VOCs concentration. A trend of the static adsorption isotherm versus the partial pressure in FIGS. 8A-8C reveals that there is a large difference between the dynamic penetrated adsorption capacity and the dynamic saturated adsorption capacity, which generally occurs at a position where the static adsorption isotherm changes steeply at the same partial pressure. This is mainly because the adsorption force is relatively weak at the adsorption conditions. Although a higher VOCs partial pressure could enhance the adsorption effect to a certain extent, the overall adsorption reserve is insufficient and needs to take a longer time to reach saturation. In the fast rising region of the adsorption isotherm (e.g., dynamic penetrated and saturated adsorption capacities at a pressure of 63.8 mbar are quite different for OMC-5.5 at 25° C. and 35° C., and for OMC-7.5 at 25° C.), the dynamic penetrated adsorption capacity is approximately 0.7 times the dynamic saturated adsorption capacity, as shown in FIG. 9. In the region where the adsorption capacity changes relatively gently in the adsorption isotherm, the dynamic penetrated adsorption capacity is approximately 0.9 times the dynamic saturated adsorption capacity.

TABLE 2
Comparison between dynamic penetrated adsorption capacity
(Q_{dp}) and dynamic saturated adsorption capacity (Q_{ds})

| | | 25° C. | | | 35° C. | | | 45° C. | | |
|---|---|---|---|---|---|---|---|---|---|---|
| Adsorbent | P | Q_{dp} | Q_{ds} | | Q_{dp} | Q_{ds} | | Q_{dp} | Q_{ds} | |
| material | (mbar) | (g/g) | (g/g) | Difference | (g/g) | (g/g) | Difference | (g/g) | (g/g) | Difference |
| ACF-2.0 | 2.0 | 0.241 | 0.249 | 3% | 0.223 | 0.230 | 3% | 0.216 | 0.224 | 3% |
| | 12.8 | 0.321 | 0.345 | 7% | 0.324 | 0.342 | 5% | 0.300 | 0.317 | 5% |
| | 63.8 | 0.224 | 0.247 | 9% | 0.180 | 0.204 | 12% | 0.180 | 0.202 | 11% |
| OMC-5.5 | 2.0 | 0.181 | 0.189 | 4% | 0.117 | 0.134 | 13% | 0.112 | 0.118 | 5% |
| | 12.8 | 0.227 | 0.250 | 9% | 0.202 | 0.224 | 10% | 0.137 | 0.172 | 20% |
| | 63.8 | 0.449 | 0.613 | 27% | 0.271 | 0.358 | 24% | 0.180 | 0.228 | 21% |
| OMC-7.5 | 2.0 | 0.095 | 0.106 | 10% | 0.064 | 0.074 | 14% | 0.062 | 0.072 | 14% |
| | 12.8 | 0.128 | 0.156 | 18% | 0.123 | 0.141 | 13% | 0.103 | 0.133 | 23% |
| | 63.8 | 0.358 | 0.560 | 36% | 0.224 | 0.271 | 17% | 0.136 | 0.163 | 17% |

The shape of the relative pressure position in the adsorption isotherm might affect a similarity between the dynamic penetrated adsorption capacity and the dynamic saturated adsorption capacity. In the fast rising region of the adsorption isotherm, a small increase in pressure could cause a large increase in adsorption. This stage also needs to take a longer time to reach adsorption saturation, resulting in a larger difference between the dynamic saturated adsorption capacity and the dynamic penetrated adsorption capacity. In the VOCs process, there is a synergistic relationship between factors such as pore size, adsorption temperature, and VOCs partial pressure. Smaller pore size, lower adsorption temperature, and higher partial pressure are all conducive to an enhancement of adsorption force. In the region where the adsorption isotherm changes gently, it is understood that under a synergistic effect of various influencing factors, the adsorption force is in excess for the pore volume and the specific surface area that could contribute to the adsorption capacity, and the adsorption equilibrium could be reached quickly. In the region where the adsorption isotherm changes steeply, the adsorption force is slightly higher than the desorption process for the pore volume and the specific surface area that could contribute to the adsorption capacity, but there is no significant difference. That is, there is no additional adsorption force reserve, and it needs to take a longer time to reach the adsorption equilibrium, resulting in a larger difference between the dynamic saturated adsorption capacity and the dynamic penetrated adsorption capacity.

Therefore, it is inferred that the proportional relationship between the dynamic penetrated adsorption capacity and the dynamic saturated adsorption capacity is related to the slope of a trend curve of the dynamic saturated adsorption capacity versus the partial pressure. Thus, the proportional relationship between the dynamic penetrated adsorption capacity and the dynamic saturated adsorption capacity and a law of its change with the slope could be obtained according to the slope of the curve at different stages. A proportional relationship equation between Q_{ds} and Q_{dp} was obtained according to a comparison between the dynamic saturated adsorption capacity Q_{ds} and the dynamic penetrated adsorption capacity Q_{dp} at a same concentration and combining an influence of a slope k of the Q_{ds} prediction curve on a difference between Q_{ds} and Q_{dp}, namely Formula 2;

$${Q}_{dp} = b \times {Q}_{ds} , \qquad Formula 2$$

where
in Formula 2,
Q_{dp} represented the dynamic penetrated adsorption capacity, in g/g;
Q_{ds} represented the dynamic saturated adsorption capacity, in g/g;
k represented the slope of the Q_{ds} prediction curve;
b satisfied an equation b=c×k+d, and b represented a proportional relationship coefficient between Q_{dp} and Q_{ds} at the same partial pressure;
c and d represented coefficients of the equation b=c×k+d, which were obtained by solving an equation set using the slope k at two different positions on the Q_{ds} prediction curve and a corresponding proportional relationship coefficient b as known numbers.

Taking a trend curve of the dynamic saturated adsorption capacity of benzene on OMC-5.5 at 35° C. versus the relative partial pressure as an example, when a pressure is about 63.8 mbar, a slope of the curve is about 0.00755, and the dynamic penetrated adsorption capacity at this time is about 0.7 times the dynamic saturated adsorption capacity. When a pressure is about 12.8 mbar, a slope of the curve is about 0.00272, and the dynamic penetrated adsorption capacity at this time is about 0.9 times the dynamic saturated adsorption capacity, as shown in FIG. 9. Two sets of data (c, d), (0.00755, 0.7) and (0.00272, 0.9), were substituted into a linear equation (b=c×k+d) to solve (c=−41.7, d=1), and to obtain a correlation equation b=1−41.7×k between the slope k and a multiple relationship b (Q_{dp}=b×Q_{ds}). Thus, a conversion relationship equation was obtained between the dynamic penetrated adsorption capacity (Q_{dp}) and the dynamic saturated adsorption capacity (Q_{ds}) of benzene adsorption at 35° C.:

$${Q}_{dp} = ( 1 - 41.7 \times k ) \times {Q}_{ds} ;$$

Q_{dp} represented the dynamic penetrated adsorption capacity, Q_{ds} represented the dynamic saturated adsorption capacity; b=1−41.7×k, and b represented a multiple relationship between Q_{ds} and Q_{dp} (b=Q_{dp}/Q_{ds}), and k represented the slope of the Q_{ds} prediction curve.

Therefore, based on a comparison of the dynamic saturated adsorption capacity (Q_{ds}) and the dynamic penetrated adsorption capacity (Q_{dp}) at a same concentration, a conversion relationship between the two capacities was obtained (Q_{dp}=(1−41.7×k)×Q_{ds}). Combined with the curve of the dynamic saturated adsorption capacity (Q_{ds}) versus the partial pressure, a curve of the dynamic penetrated adsorption capacity (Q_{dp}) versus concentration was obtained, and the dynamic penetrated adsorption capacity (Q_{dp}) of benzene at the different concentrations in dynamic adsorption at 35° C. was predicted.

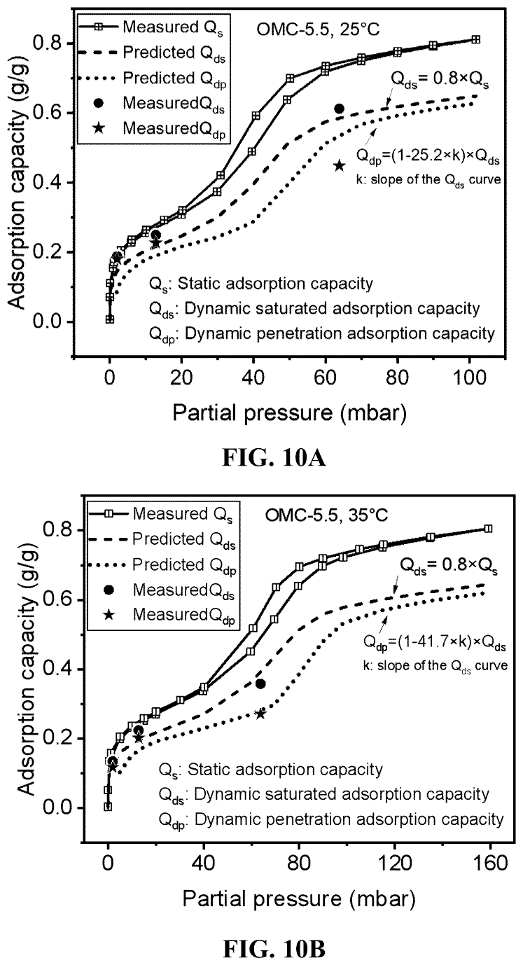

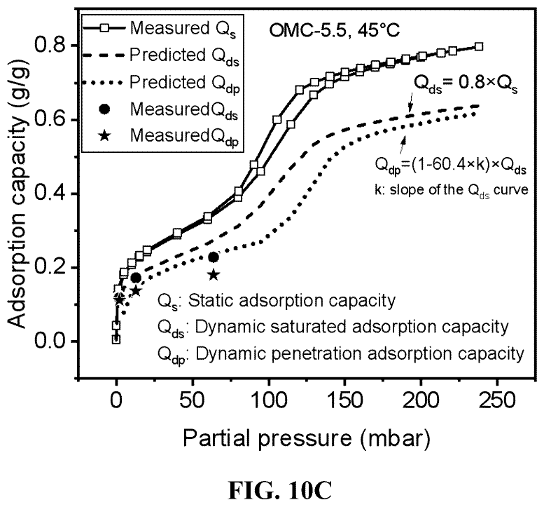

The above method was used to obtain curves of the dynamic penetrated adsorption capacity (Q_{dp}) of benzene corresponding to 25° C. and 45° C. versus the concentration. Conversion equations between the dynamic penetrated adsorption capacity (Q_{dp}) and the dynamic saturated adsorption capacity (Q_{ds}) of benzene at 25° C., 35° C., and 45° C. were as follows:

$$at 25 °C . , {Q}_{dp} = b \times {Q}_{ds} = ( 1 - 25.2 \times k ) \times {Q}_{ds} ;$$

$$at 35 °C . , {Q}_{dp} = b \times {Q}_{ds} = ( 1 - 41.7 \times k ) \times {Q}_{ds} ;$$

$$at 45 °C . , {Q}_{dp} = b \times {Q}_{ds} = ( 1 - 60.4 \times k ) \times {Q}_{ds} ;$$

where
Q_{dp} represented the dynamic penetrated adsorption capacity, in g/g; Q_{ds} represented the dynamic saturated adsorption capacity, in g/g; b represented the multiple relationship between Q_{ds} and Q_{dp} (b=Q_{dp}/Q_{ds}), and k represented the slope of the Q_{ds} prediction curve. FIGS. 10A-10C show curves of the dynamic penetrated adsorption capacity (Q_{dp}) of benzene at 25° C., 35° C., and 45° C. versus the concentration obtained by Formula 2.

### EXAMPLE 2

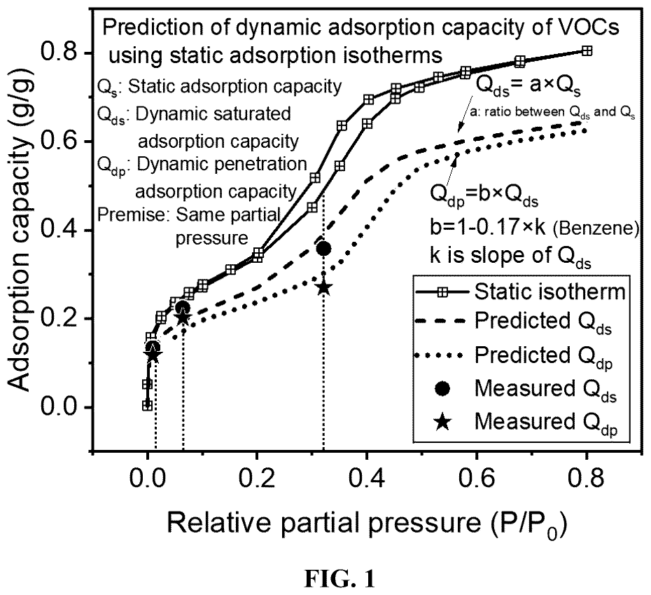

Each pressure point on the static adsorption isotherm in step (1) of Example 1, the Q_{ds} prediction curve in step (4), and the Q_{dp} prediction curve in step (5) was divided by a saturated vapor pressure at a corresponding temperature, and a curve normalized by partial pressure was obtained. Combined with an influence of a slope k₁ of a normalized Q_{ds} prediction curve, a general Q_{dp} prediction equation applicable to different adsorption temperatures, i.e., being not affected by an adsorption temperature difference, was obtained as shown in Formula 3, and a normalized prediction curve of Q_{dp} versus the partial pressure was obtained as shown in FIG. 1.

$${Q}_{dp} = {b}_{1} \times {Q}_{ds} , \qquad Formula 3$$

where
in Formula 3,
Q_{dp} represented the dynamic penetrated adsorption capacity, in g/g;
Q_{ds} represented the dynamic saturated adsorption capacity;
b₁=c₁×k₁+d₁, and b₁ represented a proportional relationship coefficient between Q_{dp} and Q_{ds} at a same relative partial pressure P/P₀, P represented a pressure on the static adsorption isotherm, and P₀ represented a saturated vapor pressure of the VOCs at a specific temperature;
k₁ represented the slope of the normalized Q_{ds} prediction curve normalized relative to the saturated vapor pressure; and
c₁ and d₁ represented coefficients of the equation b₁=c₁×k₁+d₁, which were obtained by solving an equation set using the slope k₁ at two different positions on the normalized Q_{ds} prediction curve and a corresponding proportional relationship coefficient b₁ as known numbers.

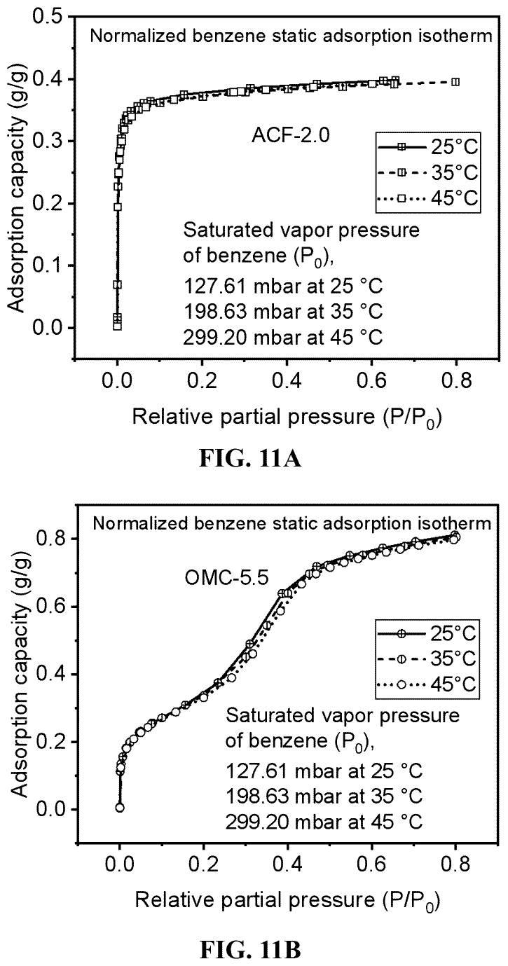

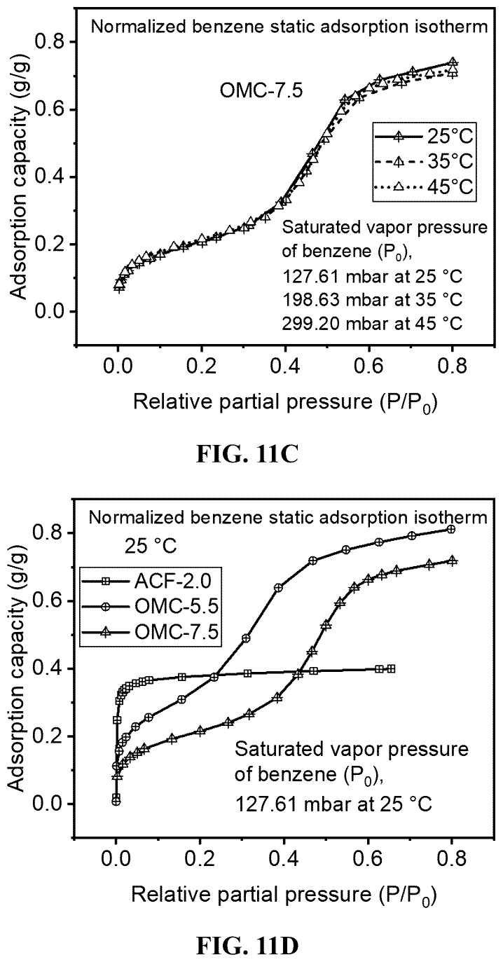

From results of the curve of the dynamic penetrated adsorption capacity (Q_{dp}) of benzene corresponding to 25° C., 35° C. and 45° C. versus the concentration obtained in Example 1, it is seen that the adsorption temperature has an important influence on the adsorption of the VOCs. Due to different adsorption temperatures, the corresponding saturated vapor pressure is different. For example, The saturated vapor pressure of benzene is 127.61 mbar at 25° C., 198.63 mbar at 35° C., and 299.20 mbar at 45° C. This might result in different coefficients in the conversion equation between the dynamic penetrated adsorption capacity (Q_{dp}) and the dynamic saturated adsorption capacity (Q_{ds}) of the same adsorbent at different temperatures. However, adsorption isotherms of the same adsorbent materials at different temperatures have similar shapes overall. P represents the pressure on the static adsorption isotherm, and P₀ represents the saturated vapor pressure of the VOCs at the specific temperature; by introducing relative pressure, that is, all pressure points on the adsorption isotherm are normalized with respect to the saturated vapor pressure at the adsorption temperature, and the saturated vapor pressure is taken as 1, a normalized curve could be obtained, as shown in FIGS. 11A-11D.

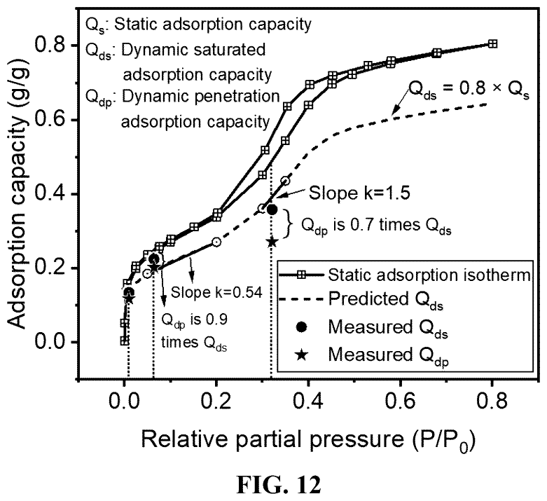

Each pressure point on the adsorption isotherm is divided by a saturated vapor pressure at a corresponding temperature to obtain an adsorption isotherm after partial pressure normalization at the corresponding temperature, such that it might be possible to predict adsorption isotherms at different temperatures using one equation. Taking a trend curve of the dynamic saturated adsorption capacity on OMC-5.5 at 35° C. versus the relative partial pressure as an example, when a relative partial pressure is about 0.3212 (partial pressure is 63.8 mbar), a slope of a curve is about 1.5, and the dynamic penetrated adsorption capacity at this time is about 0.7 times the dynamic saturated adsorption capacity. When a relative partial pressure is about 0.0644 (partial pressure is 12.8 mbar), a slope of the curve is about 0.54, and the dynamic penetrated adsorption capacity at this time is about 0.9 times the dynamic saturated adsorption capacity, as shown in FIG. 12. Two sets of data (k₁, b₁), (1.5, 0.7) and (0.5, 0.9), were substituted into a linear equation (b₁=c₁×k₁+d₁) to solve (c₁=−0.17, d₁=1), and to obtain a correlation equation b₁=1−0.17×k₁ between a slope k₁ and a multiple relationship b₁ (Q_{dp}=b₁×Q_{ds}). Thus, a conversion relationship equation was obtained between the dynamic penetrated adsorption capacity (Q_{dp}) and the dynamic saturated adsorption capacity (Q_{ds}) at the same relative pressure:

$${Q}_{dp} = {b}_{1} \times {Q}_{ds} = ( 1 - 0.17 \times {k}_{1} ) \times {Q}_{ds} ,$$

Q_{dp} represented the dynamic penetrated adsorption capacity, in g/g;
Q_{ds} represented the dynamic saturated adsorption capacity;
b₁=1−0.17×k₁, and b₁ represented a proportional relationship coefficient between Q_{dp} and Q_{ds} at the same relative partial pressure (P/P₀); k₁ represented the slope of the normalized Q_{ds} prediction curve relative to the saturated vapor pressure.

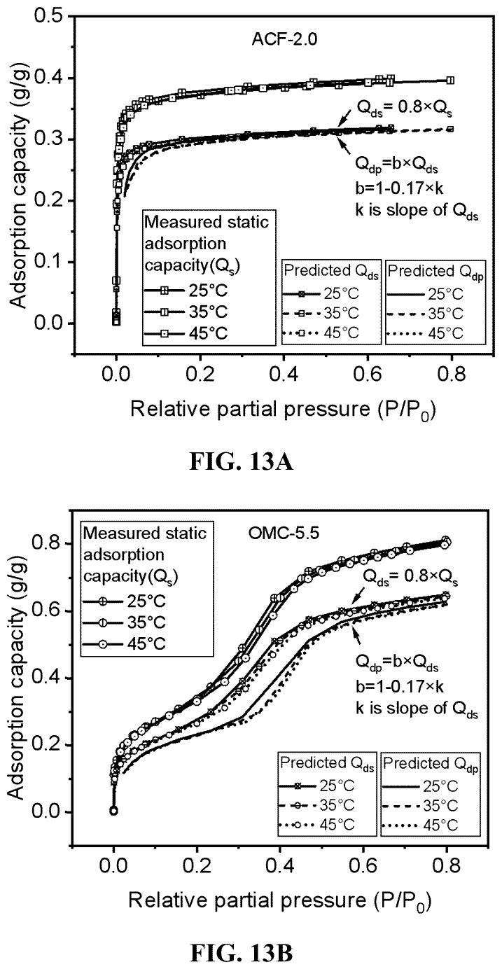

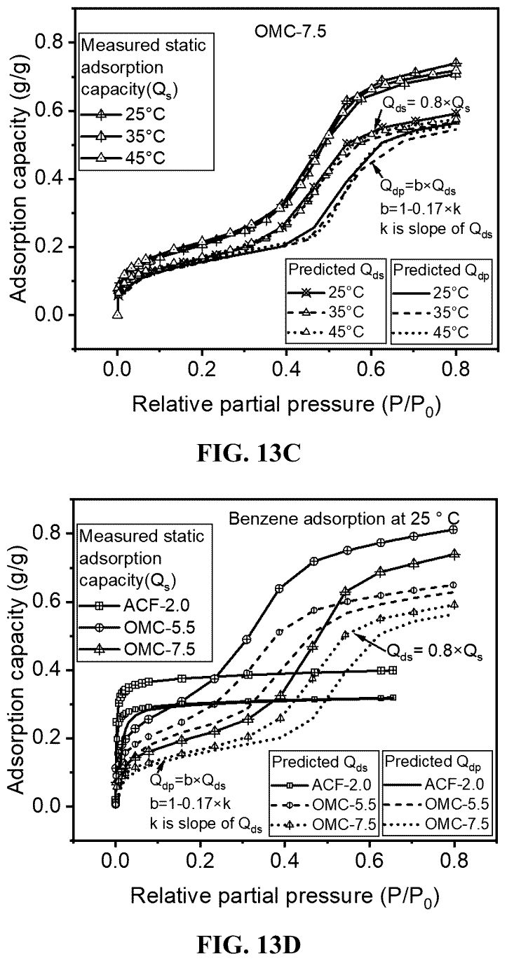

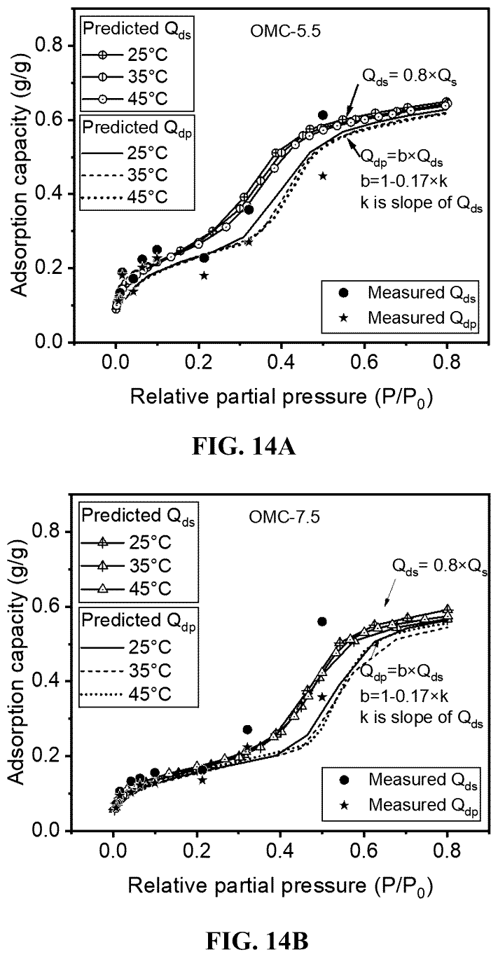

The equation (Formula 3) obtained after curves of different adsorption temperatures were normalized relative to the saturated vapor pressure at each temperature could be used to predict the dynamic penetrated adsorption capacity Q_{dp} at the different temperatures, as shown in FIGS. 13A-13D. A same equation could be used to obtain a curve of Q_{dp} versus the relative partial pressure at the different temperatures to predict the dynamic penetrated adsorption capacity (Q_{dp}) at the different temperatures and concentrations. The Q_{ds} prediction curve at the relative partial pressure and the Q_{dp} prediction curve obtained by Formula 3 were compared with the measured dynamic saturated adsorption capacity Q_{ds} and the measured dynamic penetrated adsorption capacity Q_{dp}, as shown in FIGS. 14A-14B. It is found that prediction curves could well conform to a trend of the dynamic saturated adsorption capacity Q_{ds} and the dynamic penetrated adsorption capacity Q_{dp} versus the relative partial pressure.

In summary, the method of the present disclosure can obtain the conversion relationship between the dynamic adsorption capacity and the static adsorption capacity under a premise of a same partial pressure. In the adsorption, based on an objective rule that the relative partial pressure could have an important influence on the adsorption capacity of VOCs, the static adsorption capacity and the dynamic saturated adsorption capacity at the same partial pressure are compared to find a corresponding relationship between the above two capacities. A changing trend of the adsorption capacity of adsorbent materials for VOCs in static adsorption with the increase of VOCs pressure can be used to obtain a curve of the dynamic saturated adsorption capacity versus VOCs concentration in dynamic adsorption, thereby predicting the dynamic saturated adsorption capacity of the VOCs at different concentrations. Combined with an influence of a slope k of a Q_{ds} prediction curve on a difference between Q_{ds} and Q_{dp}, a proportional relationship between Q_{ds} and Q_{dp} is revealed to achieve a prediction of the dynamic penetrated adsorption capacity at different concentrations, thereby fully reflecting the adsorption performance of the adsorbent materials for VOCs. Normalizing the partial pressure relative to a saturated vapor pressure can effectively overcome the influence of adsorption temperature differences, and obtain a general Q_{dp} prediction equation applicable to different adsorption temperatures and a Q_{dp} prediction curve versus the partial pressure at the different adsorption temperatures, thereby predicting the dynamic saturated adsorption capacity and dynamic penetrated adsorption capacity of VOCs at the different temperatures and concentrations. Due to differences in material pore structure, adsorption temperature, VOCs type, partial pressure and other factors, the conversion coefficient between dynamic adsorption capacity and static adsorption capacity in the method of the present disclosure may be different in other types of VOCs and under different adsorption conditions. The method is able to obtain corresponding coefficients and conversion relationships, thereby predicting the dynamic saturated adsorption capacity and dynamic penetrated adsorption capacity at different concentrations under the corresponding test conditions.

The method helps to improve understanding on the adsorption of gaseous pollutants and build a bridge for the conversion relationship between the static adsorption capacity and the dynamic adsorption capacity of VOCs. The method can use a static adsorption isotherm of adsorbent materials to obtain curves of the dynamic saturated adsorption capacity and the dynamic penetrated adsorption capacity versus concentration, so as to predict the dynamic saturated adsorption capacity and the dynamic penetrated adsorption capacity of the VOCs at the different concentrations. As a result, the method provide a reference for the development of efficient adsorbent materials and technologies for VOCs.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for predicting a dynamic adsorption capacity of volatile organic compounds (VOCs) at different concentrations using a static adsorption isotherm, the dynamic adsorption capacity comprising a dynamic saturated adsorption capacity Q_{ds} and a dynamic penetrated adsorption capacity Q_{dp}; wherein
a process for predicting the dynamic saturated adsorption capacity Q_{ds} comprises:
(1) providing two or more adsorbent materials with significantly different pore sizes, and then testing a static adsorption isotherm of each of the adsorbent materials on VOCs at multiple adsorption temperatures to obtain a static adsorption capacity Qₛ of the VOCs at different pressures;
(2) testing a dynamic adsorption penetration curve of each of the adsorbent materials on the VOCs at multiple concentrations at the multiple adsorption temperatures to obtain the dynamic penetrated adsorption capacity Q_{dp} and the dynamic saturated adsorption capacity Q_{ds} of the VOCs at the multiple concentrations;
(3) calculating a partial pressure corresponding to the VOCs at each of the multiple concentrations in dynamic adsorption based on a saturated vapor pressure of the VOCs at each of the multiple adsorption temperatures and a concentration-partial pressure conversion relationship, and then determining a conversion relationship equation Formula 1 between the dynamic saturated adsorption capacity Q_{ds} and the static adsorption capacity Qₛ at a same partial pressure by statistics of the dynamic saturated adsorption capacity Q_{ds} and the static adsorption capacities Qₛ at the same partial pressure,
$${Q}_{ds} = a \times {Q}_{s} , \qquad Formula 1$$
where Q_{ds} represents the dynamic saturated adsorption capacity, in g/g, Qₛ represents the static adsorption capacity, in g/g, and a represents a proportional relationship coefficient between Q_{ds} and Qₛ;
(4) obtaining the dynamic saturated adsorption capacity Q_{ds} of the VOCs at the same partial pressure in the static adsorption isotherm by combining the static adsorption capacity Qₛ corresponding to each pressure point in the static adsorption isotherm with Formula 1, and then obtaining a curve of the dynamic saturated adsorption capacity Q_{ds} versus the partial pressure according to a change trend of the static adsorption isotherm with a pressure, to obtain a Q_{ds} prediction curve, thereby predicting the dynamic saturated adsorption capacity Q_{ds} of the VOCs at the different concentrations; and
a process for predicting the dynamic penetrated adsorption capacity Q_{dp} comprises:
obtaining a proportional relationship equation Formula 2 between Q_{ds} and Q_{dp} according to a comparison between the dynamic saturated adsorption capacity Q_{ds} and the dynamic penetrated adsorption capacity Q_{dp} at a same concentration and a slope k of the Q_{ds} prediction curve,
$${Q}_{dp} = b \times {Q}_{ds} , \qquad Formula 2$$
where Q_{dp} represents the dynamic penetrated adsorption capacity, in g/g, Q_{ds} represents the dynamic saturated adsorption capacity, in g/g, b satisfies an equation b=c×k+d, and b represents a proportional relationship coefficient between Q_{dp} and Q_{ds} at the same partial pressure, k represents the slope of the Q_{ds} prediction curve, and c and d represent coefficients of the equation b=c×k+d, which are obtained by solving an equation set using the slope k at two different positions on the Q_{ds} prediction curve and a corresponding proportional relationship coefficient b as known numbers; and
obtaining a curve of the dynamic penetrated adsorption capacity Q_{dp} versus the partial pressure by combining the curve of the dynamic saturated adsorption capacity Q_{ds} versus the partial pressure with Formula 2, to obtain a Q_{dp} prediction curve, thereby predicting the dynamic penetrated adsorption capacity Q_{dp} of the VOCs at the different concentrations.

2. The method of claim 1, wherein the process for predicting the dynamic penetrated adsorption capacity further comprises:
dividing each pressure point on the static adsorption isotherm, the Q_{ds} prediction curve, and the Q_{dp} prediction curve by a saturated vapor pressure at a corresponding temperature to obtain a normalized static adsorption isotherm, a normalized Q_{ds} prediction curve, and a normalized Q_{dp} prediction curve after partial pressure normalization, and then obtaining a general Q_{dp} prediction equation Formula 3 that is not affected by an adsorption temperature difference by combining a slope k₁ of the normalized Q_{ds} prediction curve,
$${Q}_{dp} = {b}_{1} \times {Q}_{ds} , \qquad Formula 3$$
where Q_{dp} represents the dynamic penetrated adsorption capacity, in g/g, Q_{ds} represents the dynamic saturated adsorption capacity, in g/g, b₁ satisfies an equation b₁=c₁×k₁+d₁, and b₁ represents a proportional relationship coefficient between Q_{dp} and Q_{ds} at a same relative partial pressure P/P₀, P represents a pressure on the static adsorption isotherm, and P₀ represents a saturated vapor pressure of the VOCs at a specific temperature, k₁ represents the slope of the normalized Q_{ds} prediction curve relative to the saturated vapor pressure, and c₁ and d₁ represent coefficients of the equation b₁=c₁×k₁+d₁, which are obtained by solving an equation set using the slope k₁ at two different positions on the normalized Q_{ds} prediction curve and a corresponding proportional relationship coefficient b₁ as known numbers.

3. The method of claim 1, wherein each of the adsorbent materials is independently at least one selected from the group consisting of an activated carbon, a porous silica, and a molecular sieve.

4. The method of claim 1, wherein each of the adsorbent materials independently has an average pore size of 0 nm to 10 nm.

5. The method of claim 1, wherein the significantly different pore sizes indicate that average pore sizes of different adsorbent materials have a difference not less than 2 nm.

6. The method of claim 1, wherein the VOCs are selected from the group consisting of a hydrocarbon organic matter, an oxygen-containing organic matter, a halogen-containing organic matter, a nitrogen-containing organic matter, and a sulfur-containing organic matter.

7. The method of claim 1, wherein in step (1), a number of the multiple adsorption temperatures is equal to or greater than 2.

8. The method of claim 1, wherein in step (2), a concentration number of the VOCs at the multiple concentrations is equal to or greater than 2.

9. The method of claim 2, wherein each of the adsorbent materials independently has an average pore size of 0 nm to 10 nm.

10. The method of claim 2, wherein the significantly different pore sizes indicate that average pore sizes of different adsorbent materials have a difference equal to or greater than 2 nm.
